(19) United States
(12) Patent Application Publication
(10) Pub. No.: US 2017/0203060 A1
(43) Pub. Date: Jul. 20, 2017

(54) DRY POWDER NEBULIZER

(71) Applicant: MICRODOSE THERAPEUTX, INC., Ewing, NJ (US)

(72) Inventor: Mark Steven Morrison, Basking Ridge, NJ (US)

(21) Appl. No.: 15/324,989

(22) PCT Filed: Jun. 3, 2015
(86) PCT No.: PCT/US15/34050

§ 371 Date: Jan. 9, 2017

Related U.S. Application Data
(60) Provisional application No. 62/028,172, filed on Jul. 23, 2014.

(51) Int. Cl. A61M 15/00; A61M 11/00

(52) U.S. Cl. CPC A61M 15/0085; A61M 11/005; A61M 15/0065; A61M 2202/064

(57) ABSTRACT

An inhalation device comprising a drug chamber (4) for holding a pharmaceutical (3), a vibrator (2) for aerosolizing the pharmaceutical (3), and a wave reflector (6), wherein the drug chamber (4) comprises at least one inlet hole (7) and at least one outlet hole (8), so that air can be drawn into the inlet hole (7) and out from the outlet hole (8), and wherein a standing wave pattern is created by acoustic energy that is produced by the vibrator (2) and reflected back at least in part to the vibrator (2) by the reflector (6) in order to facilitate mixing and release of the pharmaceutical.

The present invention relates generally to the field of metering, packaging and delivery of pharmaceuticals and drugs. Particular utility for the present invention is found in delivery of metered and packaged dry powder medications and drugs for inhalation therapy and will be described in connection with such utility, although other utilities are contemplated, including liquid medication applications.

Certain diseases of the respiratory tract are known to respond to treatment by the direct application of therapeutic agents. As these agents are most readily available in dry powdered form, their application is most conveniently accomplished by inhaling the powdered material through the nose or mouth. This powdered form results in the better utilization of the medication in that the drug is deposited exactly at the site desired and where its action may be required; hence, very minute doses of the drug are often equally as efficacious as larger doses administered by other means, with a consequent marked reduction in the incidence of undesired side effects and medication cost. Alternatively, the drug in powdered form may be used for treatment of diseases other than those of the respiratory system. When the drug is deposited on the very large surface areas of the lungs, it may be very rapidly absorbed into the blood stream; hence, this method of application may take the place of administration by injection, tablet, or other conventional means.

It is the opinion of the pharmaceutical industry that the bioavailability of the drug is optimum when the drug particles delivered to the respiratory tract are between 1 to 5 microns in size. When the drug particles need to be in this size range the dry powder delivery system needs to address a number of issues:

(1) Small size particles develop an electrostatic charge on themselves during manufacturing and storage. This causes the particles to agglomerate or aggregate, resulting in clusters of particles which have an effective size greater than 5 microns. The probability of these large clusters making it to the deep lungs then decreases. This in turn results in a lower percentage of the drug being available to the patient for absorption.

(2) The amount of active drug that needs to be delivered to the patient may be of the order of tens of micrograms. Since current powder filling equipment cannot effectively deliver aliquots of drugs in microgram quantities with acceptable accuracy, the standard practice is to mix the active drug with a filler or bulking agent such as lactose. This additive also makes the drug “easy to flow”. In some cases this filler is sometimes called a carrier. These carrier particles are often larger than the drug particles in size. The ability of the dry powder inhaler to separate drug from the carrier is an important performance parameter in the effectiveness of the design.

(3) Active drug particles with sizes greater than 5 microns will be deposited either in the mouth or throat. This introduces another level of uncertainty since the bioavailability and absorption of the drug in these locations is different from the lungs. Dry powder inhalers need to minimize the drug deposited in these locations to reduce the uncertainty associated with the bioavailability of the drug.

Prior art dry powder inhalers (DPIs) usually have a means for introducing the drug (active drug plus carrier) into a high velocity air stream. The high velocity air-stream is used as the primary mechanism for breaking up the cluster of micronized particles or separating the drug particles from the carrier. Several inhalation devices useful for dispensing this powder form of medication are known in the prior art. For example, in U.S. Pat. Nos. 3,507,277; 3,518,992; 3,635,219; 3,795,244; and 3,807,400, inhalation devices are disclosed having means for piercing or removing the top of a capsule containing a powdered medication, which upon inhalation is drawn out of the pierced or topped capsule and into the user's mouth. Several of these patents disclose propeller means, which upon inhalation aid in dispensing the powder out of the capsule, so that it is not necessary to rely solely on the inhaled air to suction powder from the capsule.

Prior art devices such as above described have a number of disadvantages which makes them less than desirable for the delivery of dry powder to the lungs. Some of these disadvantages include:
The performance of the prior art inhalers depends on the flow rate generated by the user. Lower flow rate does not result in the powder being totally deaggregated and hence adversely affects the dose delivered to the patient.
Inconsistency in the bioavailability of the drugs from dose-to-dose because of lack of consistency in the deaggregation process.
Large energy requirements for driving the electromechanical based inhalers which increases the size of the devices making them unsuitable for portable use.
   Loss of medication from opened or topped capsules.
   Deterioration of medication in open or topped capsule due to exposure to oxygen or moisture.

The foregoing discussion of the prior art derives in part from U.S. Pat. No. 7,318,434, in which there is described a dry powder inhaler which employs synthetic jetting technology to aerosolize drug powder from a blister pack or the like. It is known that if one uses a chamber bounded on one end by an acoustic wave generating device and bounded on the other end by a rigid wall with a small orifice, that when acoustic waves are emitted at high enough frequency and amplitude from the generator, a jet of air that emanates from the orifice outward from the chamber can be produced. The jet, or so-called “synthetic jet”, is comprised of a train of vortical air puffs that are formed at the orifice at the generator's frequency. However, as described in the aforesaid '434 patent, the use of a synthetic jet to deaggregate and eject a dry-powder material from a blister pack or the like provides advantages over prior art dry powder inhalers.

More particularly, the aforesaid '434 patent provides a dry powder inhaler having a first chamber for and holding a dry powder, and a second chamber connected to the first chamber via a passageway for receiving an aerosolized form of the dry powder from the first chamber and for delivering the aerosolized dry powder to a user. A vibrator is coupled to the dry powder in the first chamber. Since jetting efficiency falls off as the aspect ratio (length to cross-section or diameter) of the passageway increases, in order to create a synthetic jet the passageway connecting the first chamber to the second chamber preferably, but not necessarily has an aspect ratio equal to at least about one, and the vibrator is energized and coupled to the first chamber so that the distance the gas moves back and forth in the passageway is at least about twice the cross-section or diameter of the passageway.

In one embodiment of the aforesaid '434 patent, the first chamber is formed in the shape of a cylinder or blister with a vibratory element either forming one wall of the chamber, or the vibratory element is formed apart from the chamber and coupled to the blister.

In a second embodiment of the aforesaid '434 patent the first chamber is formed in the shape of a horn, with a vibratory element either forming one wall of the chamber, or the vibratory element is coupled to a wall of the chamber via a column of gas.

In a third embodiment the aforesaid '434 patent the first chamber is formed in the shape of a horn, and a standing wave resonator is coupled to a wall of the chamber.

See also U.S. Pat. Nos. 7,334,577; 7,779,837 and 8,322,338, the contents of which are incorporated herein in their entirety by reference.

The blister implementation described by the aforementioned patents bears some resemblance to an inverted kettle drum, whereby a piezoelectric transducer applies acoustic energy to the open end of the chamber (i.e. drum). Small holes at the closed end provide an escape path for drug loaded in the chamber. When driven at the right frequency, as governed by dimensions of both the piezo and the chamber, a unique standing wave pattern is created that, owing to the unique shape of the chamber, conveniently places pressure anti-nodes at both ends, with a pressure node in between.

The pressure anti-node nearest the closed end of the chamber works in concert with the small holes at that end to create synthetic jets that expel drug from the chamber.

Synthetic jetting is the phenomenon by which air passing rapidly through an opening develops vortices that move away from the opening. The same thing happens in the opposite direction, at different times, such that the net air mass flow is zero. These ‘internal vortices’ (or jets) assist with mixing of powder within the chamber. However, the vortices leaving the chamber carry with them powdered drug, which leaves the chamber and does not return. These are the particles available for patient inhalation.

In one aspect of the invention there is provided an inhalation device comprising a drug chamber for holding a pharmaceutical;
a vibrator for aerosolizing said pharmaceutical; and
a wave reflector, wherein the drug chamber comprises at least one inlet hole and at least one outlet hole; and
wherein the device is configured such that a standing wave pattern is created by acoustic energy that is produced by the vibrator and reflected back at least in part to the vibrator by the reflector so that air can be drawn into the inlet hole and out from the outlet hole.

In one embodiment, the acoustic waves are produced by a piezoelectric transducer.

With the present invention it is possible to deliver a drug for inhalation in a simple and reliable way with a device which is low cost yet capable of effective delivery. It also enables a device which is compact and with low power requirements.

Examples of the present invention will now be described with reference to the accompanying drawings, in which:

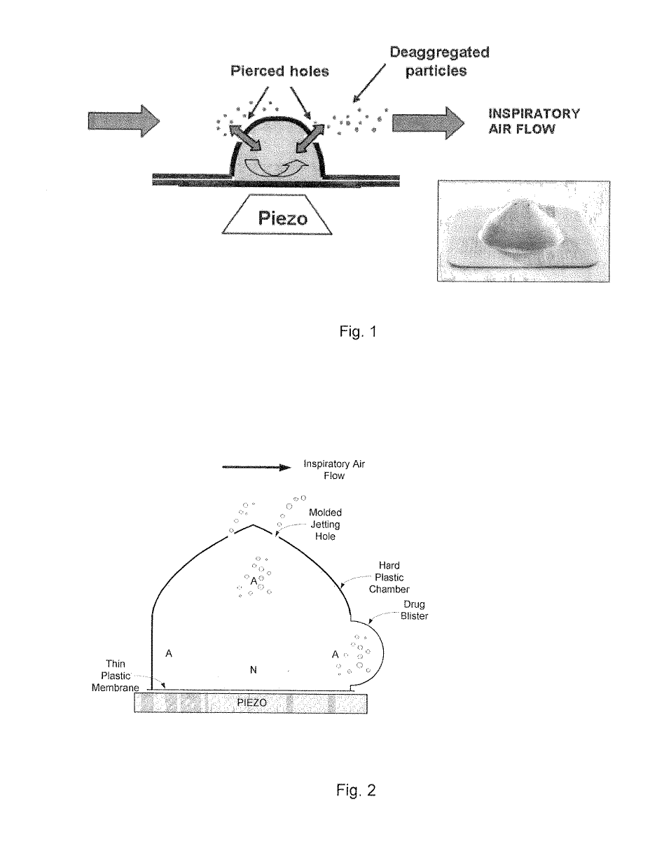

FIGS. 1 and 2 are schematic side views of examples of prior art arrangements; and

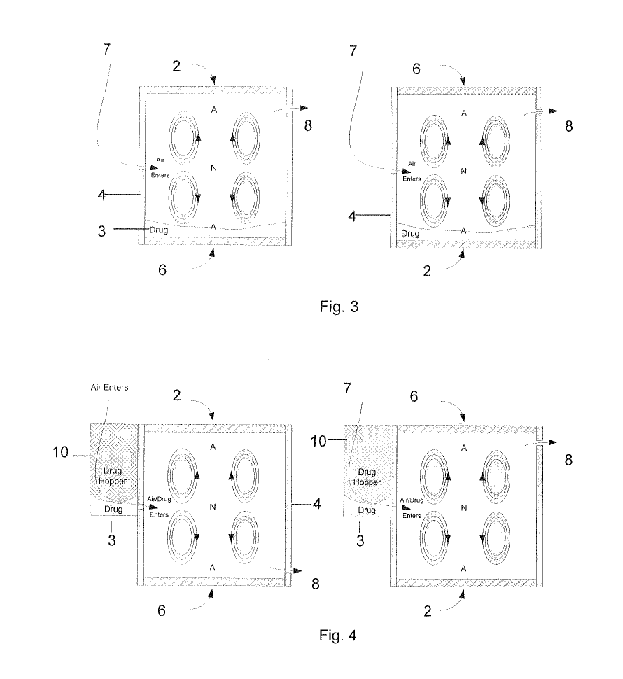

FIGS. 3 and 4 are schematic side views of components of a device according to the invention.

Referring to FIG. 1, a known design uses a special dome shaped drug blister as the chamber. This requires a special piercing tool to create the jetting holes just prior to use. In this case, the piezo is placed in contact with the lidding material of the sealed blister, vibrating the bottom of the blister and causing direct agitation of the drug powder within. In this capacity, the piezo 1) creates the acoustic waves that result in synthetic jetting, and 2) deagglomerates the drug resting on the lid material by direct vibration.

More recently, an alternative has been designed, and is a drug delivery system comprising a dose chamber coupled to a vibrating device as described in U.S. application Ser. No. 12/985,158, the contents of which are incorporated herein by reference. In an embodiment described in the '158 application, an inhaler is provided with a combined reservoir and dosing chamber configured to receive multiple doses of a pharmaceutical material. As before, the dosing chamber is coupled to a vibration device for aerosolizing the pharmaceutical, and delivering aerosolized pharmaceuticals to the patient.

Even more recently, the hard dosing chamber described in the '158 patent has been modified to include a thin membrane that serves to both seal off the dosing chamber as well as couple the chamber to the vibrating device as illustrated in FIG. 2 (note that A stands for pressure antinode, and N stands for pressure node).

As can be seen, a thin plastic film now covers the open end, through which the piezo applies acoustic energy. Small jetting holes are molded into the chamber, replacing those created in the original design by way of piercing. In this case, the drug blister itself has been relocated to the side of the chamber, where its contents are delivered to the chamber through a small opening in the chamber wall, as a result of the lidding material being peeled back. In this position, the opening of the blister is placed in close proximity to a pressure antinode (A) on the outer circumference of the chamber. The transport of drug from the blister to the chamber is thought to be facilitated by pressure variations at the antinode as well as direct vibration of the piezo coupled into the blister by way of the surrounding structure, which is in communication with the piezo.

It should be noted that all of the previous descriptions employ synthetic jetting to transport powdered drug to the patient for inhalation. The present invention, on the other hand, uses using acoustic streaming to disperse the powder pharmaceutical. Acoustic streaming is the phenomenon by which sound travelling through a medium imparts momentum to that medium, causing it to move. One example is so called ‘Rayleigh Streaming’, which can be demonstrated using a 40 kHz piezo transducer. For example, if such a transducer is driven at sufficiently high amplitude facing into a closed tube with a suitable reflector at the opposite end, it is possible to displace powders within the tube. Surprisingly, this effect is more than adequate to aerosolize deagglomerated fine powders. This effect may also deagglomerate some drug pellets, as would be understood by a person skilled in the art.

As shown in FIG. 3, the inventors have surprisingly found that if a reflector 6 is introduced, such that most of the waves are returned directly to the transducer, it is possible to create a standing wave pattern within the chamber. If such a chamber is a simple tube shape, with the piezo 2 closing off one end, and a hard reflector at the other, pressure nodes and antinodes are developed at very specific locations. By placing holes 7, 8 in the chamber walls 4 at these locations, a node serves as a pump inlet while a pressure antinode serves as a pump outlet. If driven with sufficient acoustic energy, acoustic non-linearities form within the sound field, causing a pressure swing at the anti-node to become asymmetric, resulting in a pressure differential that is sufficient to create flow. Note that the piezo 2 can be placed at either end of the tube, but better deagglomeration is possible with the piezo placed at the bottom of the tube where it can assist with deagglomeration by means of direct vibration of the drug powder.

When dry powder is introduced into the system, which can be done by any convenient means (not illustrated), the powder may follow the path of air being pumped through the system. This is shown in FIGS. 3 and 4 where an example of a dry powder nebulizer based upon the “standing wave pump” is illustrated. In this scenario, the piezo 2 (PZT) serves not only to create the required acoustic waves, but also to actively vibrate the powdered drug 3. We note that this pumping action does not involve synthetic jetting.

An alternative configuration is illustrated in FIG. 4. In this case, drug 3 is contained inside a hopper 10 that is in fluid communication with a pressure node. Drug is “metered” out of the hopper 10 into the standing wave pump where it finds its way out the pressure antinode at the bottom. In this case, vibrations of the piezo 2 couple directly into the drug hopper, agitating particles and causing them to move into the chamber.

Fine particles can become trapped within pressure nodes, coming to rest under the force of gravity at the junction of that node and the antinode directly below it. This may otherwise be considered disadvantageous but, on the contrary, it is possible to exploit this effect for the purpose of metering out discrete packets of powdered drug. This would be particularly advantageous for the nebulizer illustrated above, which does not have drug blisters to provide such metering. Particles can be trapped in small packets and then caused to move toward some convenient exit point where they may be released from the system. It is possible to achieve this by changing the frequency of piezo operation, while being careful not to operate the piezo off resonance, where output would otherwise drop considerably. Such embodiments are included in the present invention.

In another embodiment, a further means of accomplishing the same thing is to use a reflector 6 with acoustic impedance that results in partial reflection, in which case some of the acoustic energy travels through the reflector, and some is reflected. The weaker reflected waves can provide a different, possibly changing, interference pattern that causes the nodes and anti-nodes to move. The advantage of this is that there is no requirement to change frequency on the fly, something that greatly simplifies the device. Also, this can facilitate using a drug reservoir approach, as opposed to a blister approach, providing further benefit by eliminating the drug strip, motor and related sensor. Of course a motor might still be required in order to bring the drug within proximity of the (acoustic) driving source.

It should be noted that only particles of a certain size tend to become trapped within the pressure nodes. Those too heavy to be supported by the acoustic field fall back to the drug load where they can be further agitated. Those that are light enough to be supported can be used for inhalation.

It should be noted that although a drug hopper is shown in FIG. 4, any means of delivering powder to the chamber could be used instead, including but not limited to drug blisters.

It should be understood that the foregoing detailed description and preferred embodiments are only illustrative of inhalation devices constructed in accordance with the present disclosure. Various alternatives and modifications to the presently disclosed inhalers can be devised by those skilled in the art without departing from the spirit and scope of the present disclosure.

1: An inhalation device comprising a drug chamber for holding a pharmaceutical;
a vibrator for aerosolizing said pharmaceutical; and
a wave reflector, wherein the drug chamber comprises at least one inlet hole and at least one outlet hole; and
wherein the device is configured such that a standing wave pattern is created by acoustic energy that is produced by the vibrator and reflected back at least in part to the vibrator by the reflector so that air can be drawn into the inlet hole and out from the outlet hole.

2: The device of claim 1, wherein the vibrator comprises a piezoelectric transducer.

3: The device of claim 1, wherein the drug chamber is tube-shaped.

4: The device of claim 1, wherein the vibrator and reflector are arranged at opposing ends of the chamber.

5: The device of claim 1, wherein the pharmaceutical comprises a dry powder.

6: The device of claim 1, wherein the at least one inlet hole is located adjacent to a node of the standing wave pattern.

7: The device of claim 1, wherein the at least one outlet hole is located adjacent to an antinode of the standing wave pattern.

8: The device of claim 1, wherein acoustic energy produced by the vibrator is sufficient to create a flow within the chamber capable of carrying the pharmaceutical out of the outlet.

9: The device of claim 1, wherein the pharmaceutical is contained in the chamber.

10: The device of claim 1, wherein the pharmaceutical is contained in a reservoir in communication with the chamber.

11: The device of claim 10, wherein the reservoir is located adjacent to an antinode of the standing wave pattern.

12: The device of claim 11, wherein at least one node and at least one antinode of the standing wave pattern are arranged such that a discrete amount of pharmaceutical may be metered out of the reservoir and come to rest at a junction between the node and antinode.

13: The device of claim 1, wherein the reflector has an acoustic impedance such that a portion of the acoustic energy produced by the vibrator is transmitted through the reflector.

14: The device of claim 13, wherein the transmission of a portion of the acoustic energy through the reflector creates an acoustic energy interference pattern with shifting nodes and antinodes.

15: A method for nebulizing a pharmaceutical, said method comprising the steps of:
providing a drug chamber comprising at least one inlet hole and at least one outlet hole;
arranging a vibrator and a reflector at opposing ends of the drug chamber;
directing acoustic energy produced by the vibrator to the reflector such that at least a portion of the acoustic energy is reflected back to the vibrator;
introducing a dry powder pharmaceutical into the drug chamber;
wherein the dry powder pharmaceutical is aerosolized into a standing wave pattern created by the reflected acoustic energy.

16: The method of claim 15, wherein the drug chamber is tube-shaped.

17: The method of claim 15, wherein at least one inlet hole is adjacent to at least one node created by the standing wave pattern and at least one outlet hole is adjacent to at least one antinode created by the standing wave pattern.

18: The method of claim 1, wherein the pharmaceutical is introduced into the drug chamber from a reservoir in communication with the chamber.

19: The method of claim 18, wherein the reservoir is located adjacent to at least one antinode created by the standing wave pattern.

20: The method of claim 18, wherein a discrete amount of pharmaceutical is metered out of the reservoir into a junction between at least one node and at least one antinode created by the standing wave pattern.

21: The method of claim 1, wherein a portion of the acoustic energy is transmitted through the reflector.
